(19) United States
(12) Patent Application Publication
(10) Pub. No.: US 2025/0006354 A1
(43) Pub. Date: Jan. 2, 2025

(54) DETECTING EMOTIONAL STATE OF A USER

(71) Applicant: Earkick, Inc., Wilmington, DE (US)

(72) Inventors: Veronique LARCHER, Zürich (CH); Karin Andrea STEPHAN, Wädenswil (CH); Herbert BAY, Wädenswil (CH); Srivignesh RAJENDRAN, Mountain View, CA (US)

(73) Assignee: Earkick, Inc., Wilmington, DE (US)

(21) Appl. No.: 18/704,667

(22) PCT Filed: Oct. 25, 2021
(86) PCT No.: PCT/US2021/056441

§ 371 Date: Apr. 25, 2024

(51) Int. Cl. G16H 40/40; G16H 20/70; G16H 40/67; G16H 50/20

(52) U.S. Cl. CPC G16H 40/40; G16H 20/70; G16H 40/67; G16H 50/20

(57) ABSTRACT

The invention relates to a detection device (10) for detecting the emotional state of a user. The detection device (10) comprises a processing unit (1) for processing data, in particular input data, a main data storage unit (2) for storing data, in particular input data and/or data processed by said processing unit and a connecting element (3a, 3b) for connecting the detection device (10) to an interface device (50), in particular a mobile phone or a tablet, and/or a recording device (7a, 7b). The detection device (10) is adapted to be calibrated to said user by use of the processing unit (1) and calibration data. In particular, the calibration data is at least one set, preferably five sets, of audio and video data of said user. The processing unit (1) is adapted to analyse input data based on said calibration. In particular, the processing unit (1) is adapted to compare input data to calibration data and to calculate the nearest approximation of the input data and the calibration data.

This application is a National Stage completion of PCT/US2021/056441 filed Oct. 25, 2021.

### FIELD OF THE INVENTION

The present invention relates to a detection device and a system for detecting the emotional state of a user, a computer-implemented method for detecting the emotional state of a user and a computer program product for said method.

### BACKGROUND OF THE INVENTION

Such detection of the emotion can help a person to understand their emotions and their emotional state better, which is particularly indicated for a person who is living more detached from their emotions living in a fast paced world. It is also possible to analyse public appearance and/or voice and get a neutral feedback and thus use the invention in a business context.

Emotions are an essential part of human nature as humans experience hundreds of emotions continuously. Such emotions are for example “tired”, “happy”, “relaxed”, “fear”, “alarmed”, “excited”, “astonished”, “delighted”, “pleased”, “content”, “serene”, “calm”, “sleepy”, “bored”, “depressed”, “miserable”, “frustrated”, “annoyed”, “angry”, “afraid” or “neutral”. Some emotions are typically unpleasant to experience, such as “fear” or “sadness”. These types of emotions are considered to be negative emotions. Some emotions are typically pleasurable to experience, such as “joy” or “happiness”. These types of emotions are considered to be positive emotions.

Emotions such as “joy” and “happiness” give humans a benefit to social development and even physical health. However, even negative emotions such as “fear,” “anger,” and “sadness” have their uses in daily life, as they stimulate people to take actions that increase their chances of survival and promote their growth and development as human beings.

The presence of mainly negative or positive emotions presents indicators of an overall emotional state. For example, emotions such as fear, anger, sadness and worry represent indicators of a negative emotional state. Such a negative emotional state may be present after the death of a loved one. However, if such a negative emotional state is excessive, irrational and ongoing, this may indicate the presence of mental illness symptoms. On the other extreme, the presence of mainly positive emotions such as happiness, joy and hope, a positive emotional state, are associated with greater resilience against mental illness.

The emotions are displayed in humans in different ways, such as their voice, facial expression, posture, heart rate, blood pressure, sweating etc. The indicator for emotions in humans in their voices is not just words, but also in various features displayed in the tonality of the voice.

U.S. Pat. No. 9,330,658 B2 discloses a method of assessing speaker intent. The method uses among other things parameters of the voice to recognize stress of the speaker. However, the method does not take into account the individuality of the display of emotions.

Further, in the state of the art, various sensors for sensing a user are already known, such as microphones, cameras, etc., as well as programs for extracting features of data recorded by said sensors. A major challenge is the preparation of raw data obtained by a sensor, such as a microphone, which is natural (as opposed to “simulated” or “semi-natural”). Known microphones provide data with a lot of background noises and unclear voices, when used in the day-to-day life of a user. This leads to the need of sophisticated methods for speech denoising in the state of the art, such as CN105957537B.

In the state of the arts, large data bases are used as training material for self-supervised learning computer structures for detecting emotions. Thus, the training of the self-supervised learning structure allows for a general recognition of emotions of humans. However, emotions are highly individualistic, every culture and every individual displays emotions in their own way. Therefore, a problem of the state of the art is that the analysis of the display of emotions of individuals is misinterpreted or imprecise.

Further, studies have shown that reliance on one type of sensor or type of data has its drawbacks as such a device or method for analyzing emotions is also imprecise. Thus, it is preferable to analyze data of different types of sensors, such as a camera and microphone.

However, the use of more than one type of sensor data provides further obstacles: Training a self-supervised learning program structure for analyzing both audio and video requires large volumes of several tens of thousands of annotated audio-visual clips, especially for a device adjusted to a specific user.

Thus, it is important for the self-supervised learning program structure to analyse data of the user to which it should be calibrated to, without the use of a large quantity of data and which has to observe natural data of the user.

### SUMMARY OF THE INVENTION

Therefore, the problems to be solved by the present invention is to eliminate disadvantages of the state of the art and present a detection device for detecting an emotional state of a user, a system for detecting an emotional state of a user and a method for detecting an emotional state of a user, as well as a computer program product for said method, which are more precise in the recognition of the displayed emotion of an individual, especially without the use of large quantities of data of the individual, and provide an easy and robust way for using raw data, as well as provide an easy and quick way to detect emotions of the user and the emotional state of a user.

The problems are solved by a detection device for detecting an emotional state, a system for detecting an emotional state, a computer-implemented method for detecting an emotional state and a computer program product for the method according to the independent claims.

The above mentioned problems are in particular solved by a detection device for detecting the emotional state of a user. The detection device comprises
A processing unit for processing data, in particular input data,
A main data storage unit for storing data, in particular input data and/or data processed by said processing unit,
A connecting element for connecting the detection device to an interface device, in particular a mobile phone or a tablet, and/or a recording device.

The detection device is adapted to be calibrated to said user by use of the processing unit and calibration data, in particular the calibration data is at least one set, preferably at least five sets, of audio and video data of said user. The processing unit is adapted to analyse input data based on said calibration. In particular, the processing unit is adapted to compare input data to calibration data and to calculate the nearest approximation of the input data and the calibration data.

Such a detection device allows for an easy and precise recognition of the emotions displayed by a certain user. As the device is adapted to the user, the detection device recognizes the differences in the display of emotions due to individuality, culture etc. Additionally, the personal data in the calibration data set of the user are stored on a separate device, independent from other accessible devices such as mobile phones or tablets.

An emotional state is defined in this document as a tendency of negative or positive emotions recorded in a user over a certain amount of time, such as three weeks.

Sets of audio and video data refer to matching audio and video data, as in being recorded at the same time. In this case, the sets may be video and audio of a user talking. Studies have shown that analyzing both audio and video information of the user provides a better understanding of an individual user as just one or the other.

Calibration data refers to data of the user recorded during calibration of the detection device and used to calibrate the detection device. Using both audio and video data as calibration data has been shown to be much better for a holistic understanding of a user's emotions in both tonality and facial expressions and thus leads to a more precise calibration of the detection device.

Input data refers to data recorded during an analysis phase, the input data is thus to be analyzed as part of the detection of the emotional state of the user. The input data may be at least one of audio data and video data, preferably a set of audio and video data. The expression “at least one of A and B” stands for “A and/or B” in this document.

The connecting element may be adapted to transfer calibration data and/or input data to the detection device. The connecting element may be adapted for transporting data, in particular calibration data and input data, to the main data storage unit. The main data storage unit may be in a data connection to the processing unit.

Especially, the detection device may be adapted to send calibrations instructions to the user via the connecting element. The calibration instructions may comprise instructions to record themselves, in particular with a camera and/or microphone, talking about their day and/or remembering events evoking a certain emotion. Thus, the user may be instructed to remember an event, where they were angry, sad, scared, happy, calm, tired, excited etc. allowing the detection device to calibrate itself to the user. The user may be instructed to tag calibration data with an emotions tag. An emotions tag is a tag which denotes the tagged data as disclosing an emotion such as “happy”, “angry”, “scared” etc. There may be included an option to denote that the user cannot remember an event provoking a certain emotion, such as “happiness” or “sadness”.

In case of the user already being depressed, the user may not be able to remember the last time they felt happy, in severe cases, the user may not be able to remember other feelings. In case of the user having an anxiety disorder, they may be too worried and afraid to remember other feelings. This would complicate the calibration, as the user may not be able to tag calibration data as asked. Thus, such an option may help with a risk calculation for a negative emotional state.

The detection device may be adapted to conduct an initial assessment, especially via a questionnaire. Especially, the detection device may be adapted to calculate an initial risk for mental illness such as depression or anxiety, in particular based on responses of the user to instructions of the detection device. Such an initial assessment would allow calculating a risk despite the difficulties of calibrating the device to the user.

The detection device may comprise an energy supply element such as a port for connecting to a power source. The energy supply element and the connecting element may be combined.

The detection device may comprise a power source such as a battery, the battery may be rechargeable.

The detection device may comprise a locating element, especially a GPS element, for finding the detection device and/or for registering the location of the recording of input data. This allows for detecting the location of the detection device in case of loss or theft and/or for analyzing the reaction of the user to certain locations. It may be important to realize, that a certain location may be worsening the emotional state of the user. Otherwise, finding the location of the detection device may be important, as the detection device contains personal data of the user.

The detection device may comprise a fixing element such as a keyring or other elements for securing the detection device to a bag or to trousers.

Preferably, the processing unit is adapted to process said input data and said calibration data, in particular by preparing the input data and the calibration data, and extracting emotional features of said input data and calibration data.

This allows for an easy use of the detection device. In particular, the detection device may be adapted to find the most distinguishing emotional features of the calibration data and the input data.

Preparing may include at least one of
Extracting bounding boxes to detect frontal faces, in particular in every video frame,
Cropping video data, especially the face bounding boxes,
Resizing video data, especially the face bounding boxes,
Splitting audio data into time windows of a certain length, especially time windows of shorter than 5 seconds, especially time windows of 1.2 seconds,
Splitting audio data, especially time windows of said audio data, into frames, in particular into frames of 25 ms and especially with 10 ms of overlap.

Extracting emotional features may include at least
Extracting pixels, especially pixels regarding facial expressions or posture, from video data, especially from prepared and/or cropped and/or resized video data.

More features may include:
Detect number of laughs, coughs, clearing throat, ‘ems’, etc. from voice.
Blinking rate, pupil size, eye movements including saccades from video
Step detection, tremor detection, gait analysis, body posture estimation using accelerometers
Typing time stamps for each character from the keyboard in order to detect fatigue, concentration problems etc. of the typer.
Extracting humidity of the skin on the ear using sensors in the head phone.
Extracting air humidity and detect effect of environmental humidity on the emotional state of the user
Extracting respiratory rate using microphone in the hearables.
Using EKG data from fitness monitor or hearables or other device including at least one parameter of
   Heart rate
   Heart rate variability
   [symbol] Ischemia
   [symbol] Heart attack
Extracting audio features from audio data, especially of a time window and/or a frame of said time window, the emotional features may comprise at least one of
   [symbol] A fundamental frequency of the voice
   A parameter of the formant frequencies of the voice
   Jitter of the voice
   Shimmer of the voice
   Intensity of the voice

Any one of these features may help in calculating the emotion the user displays in the data recorded. For example, a lowering mobility/activity score could be indicative of growing negative emotions.

Especially the voice provides a lot of information on the emotional state of the user:

Stress leads to tension in the larynx and thus exerts pressure on it, which in turn leads to a squeezed voice sound and possible voice failure. The reason for this is the undersupply of oxygen to the vocal cords. A stressed voice often sounds shaky and soft and breaks when speaking.

The emotional features of the voice are the fundamental frequency, the parameters of the formant frequencies, the jitter, the shimmer and the intensity.

The fundamental frequency is the lowest frequency of a periodic waveform. The fundamental frequency is associated with the rate of glottic rotation and is considered a prosody feature. Prosody features are features of a speech that are not individual phonetic segments such as vowels and consonants, but are properties of syllables and larger units of speech. The fundamental frequency will change in the case of emotional arousal and more specifically its value in the case of anxiety will increase. The fundamental frequency parameter is the most affected parameter from anxiety.

The formant frequency is the broad spectral maximum frequency that results from an acoustic resonance of the human vocal tract. The parameters of the formant frequencies may be extracted as emotional features. The parameters are mel-frequency cepstral coefficients (MFCC), linear prediction cepstral coefficients (LPCC) and wavelet coefficients.

Each formant is characterized by its own center frequency and bandwidth, and contains important information about the emotion. For example, people cannot produce vowels under stress and depression, and the same is true in the case of neutral feelings. This change in voice causes differences in formant bandwidths. The anxious state caused changes in formant frequencies. That is, in the case of anxiety, the vocalization of the vowels decreases. Thus, analyzing these features present an easy and quick way of detecting an emotion in audio data of the user.

MFCC are the coefficients that collectively make up the mel-frequency cepstrum. The mel-frequency cepstrum is a representation of the short-term power spectrum of a sound. “Mel” describes the equally spaced frequency bands on the mel-scale, which approximates human recognized pitch. The basis for generating the MFCC is a linear modeling of the voice generation.

LPCC are the cepstral coefficients derived from the linear prediction coefficients, they are the coefficients of the Fourier transform illustration of the logarithmic magnitude spectrum.

A wavelet is a waveform of effectively limited duration that has an average value of zero.

Jitter is the micro variation of the fundamental frequency. Jitter is also influenced by gender, which affects jitter parameter by 64.8%. Thus, in the calibration gender has to be accounted for.

Shimmer is the median difference in dB between successive amplitudes of a signal, the amplitudes being the median distance between to frequency maxima.

Sound intensity is the power carried by sound waves per unit area in a direction perpendicular to that area.

Increases in jitter, shimmer and intensity are observed in the anxious state and the sound intensity is irregular.

A combination of multiple of these features allow for a more precise reading of the emotions displayed by the user.

Additionally to the extraction of emotional features, the analysis may comprise the extraction of external features, as in features not pertaining to the user themselves. These features may include but are not limited to ambient sounds, location, time, calendar entries etc.

Especially, the processing unit may be programed to process data, especially video data and/or audio data, for analysis, by searching for differences in audio and/or video data in a calibration phase. The processing unit may be programmed to analyse the audio data, especially audio data regarding to the voice of the user, in regards to speech detection features, such as frequencies, jitter, shimmer, etc. The processing unit may be programmed to analyse video data in regards to the pixels, especially pixels of the face of the user, especially pixels showing movements of the lips and the jaw. The detection device may be programmed to store the processed data, and delete raw data of the user.

The detection device may be adapted to calibrate itself to the user by use of raw calibration data, especially raw audio-visual data of the user.

Raw data refers to data as it is recorded without further processing and/or preparing of the data such as denoising the data.

The detection device may be adapted to analyze raw input data.

Especially, the raw data is not stored after the preparation; it suffices to store the prepared data or the extracted features of said data.

The processing unit may be adapted to calculate an approximate emotion displayed by the user in the input data, based on the calibration. The processing unit may be adapted to calculate an emotional state of the user based on multiple, in particular more than ten, input data. Emotions detectable are emotions such as “tired”, “happy”, “relaxed”, “fear”, “alarmed”, “excited”, “astonished”, “delighted”, “pleased”, “content”, “serene”, “calm”, “sleepy”, “bored”, “depressed”, “miserable”, “frustrated”, “annoyed”, “angry”, “afraid”, “neutral”, etc.

The detection device may be programmed to compare the calculation data to an emotions data bank. The detection device may be adapted to tag input data based on the calculated nearest approximation with an emotions tag and/or a risk tag. A risk tag is a tag which denotes the tagged data as disclosing an emotion, which provides a higher or lower risk for a negative emotional state. Negative emotions such as “sad”, “scared” and “angry” are indicative of a higher risk for a negative emotional state. Positive emotions, such as “happy”, “joyous”, and “hopeful” are indicative of a lower risk for a negative emotional state.

For the calibration as well as the analysis the processing unit is programmed to process data and extract features of the data. The data refers to data of the user recorded by a recording device, the recording device may be a camera, a microphone, a photoplethysmographic sensor, a galvanic skin response sensor and an electroencephalogram sensor, a pulse sensor, a blood pressure meter and similar sensors.

Especially, the processing unit may comprise a computer program structure for calibrating the detection device to a user. The computer program structure may comprise a calibration mode. In the calibration mode, the processing unit may be programmed to send instructions to a user, such as to record themselves, especially to record themselves recounting their day and/or remembering events evoking certain emotions, and to tag the records with an emotions tag. Further, the processing unit may be programmed to process the recorded calibration data and store the processed data as reference data.

The detection device may be programmed to calculate a risk of mental illness based on multiple approximations of input data to negative emotions over a period of time of at least four weeks.

In particular, the processing unit may be adapted to process said calibration data into reference data.

Reference data refers to data, which is prepared in such a way, as to allow the processing unit to compare input data to it during analysis. The reference data may comprise at least one of processed video data of the user and processed audio data of the user. The reference data comprises an emotions tag: In the calibration mode, at least one of video data and audio data is tagged by the user to disclose a certain emotion such as anger, sadness, happiness etc.

The processing of the calibration data into reference data may comprise preparing raw recorded calibration data, tagging prepared calibration data and/or parts of the prepared calibration data with an emotions tag by the user, extracting distinguishing features of calibration data and storing the prepared and/or tagged calibration data or parts of the calibration data, and/or the extracted distinguishing features, by the processing unit, in the calibration mode. Especially, the processing unit may be programmed to lead the user through calibration, in particular on input of the user to start the calibration phase. The processing unit may be programmed to instruct the user to record themselves and/or recording the user after instructing the user to remember certain events, emotions etc. The processing unit may be programmed to tag recorded data during a calibration phase as a reference emotion, and/or instruct the user to tag recorded data during the calibration phase as a reference emotion. The processing unit may be programmed to extract feature vectors of the calibration data and store the feature vectors as reference data, wherein each feature vector may represent a data reference point. Especially, the processing unit may be programmed to compare a first calibration data tagged as a first emotion to a second calibration data tagged as a second emotion. The processing unit may be programmed to extract differentiating features of the first calibration data and the second calibration data.

Especially, the detection device may be programmed to analyze the calibration data with regards to the above mentioned emotional features and to calculate the most relevant distinctions of expressions of the user. For example, a user may not show much emotion in the facial expressions such as compressions of the mouth or widening of the eyes, but they may show a lot of differences in the jitter in their voice. The detection device may be programmed to analyze the input data with regards to the calculated most relevant distinctions of expressions in the user and calculate a commonality of the emotional features of the input data to the calibration data. The detection device may be programmed to supplement this analysis with less relevant distinctions of expressions of the user, especially in case the calculated emotion is inconclusive.

The detection device may be programmed to instruct the user in the use of recording devices, the calibration of the detection device and the tagging of calibration data. Especially, the detection device may be programmed to instruct the user to record themselves with a certain sensor, which records the most relevant distinction of expressions of the user.

The detection device may be programmed to screen data recorded by the user for emotional features associated with negative emotions.

The detection device may especially be programmed to screen data recorded by the user for audio features as mentioned above, in particular acoustic audio features pertaining to the sound of the voice and transformations thereof, and for content features of what is said, in particular for predetermined words.

The detection device may recognize audio features of the voice, such as mentioned above, and content features of what is said. The audio features and the content features may be used as emotional features for determining the emotional state of the user. Especially, both audio features and content features may be used in combination for determining the emotional state of the user.

This proves especially advantages, as this allows for a very precise analysis of the emotional state of the user.

For this, the detection device may recognize words used in speech and writing. Especially, the detection device may be adapted to recognize specific words associated with emotions, in particular predetermined words such as mentioned below.

When experiencing more negative emotions, persons tend to use more negative words, such as “afraid”. “lonely”, “sad”, “unhappy”, “worried”, “stressed”, and absolutes, such as “always”, “never”, “completely” or intensifiers such as “very” or “really”. Further, persons preoccupied with their own problems or more stressed tend to talk more about themselves, and use more words such as “I”, “me” and “myself”. On the flip side, someone positive might use words such as “great”, “amazing” etc.

Especially, the detection device may be programmed to recognize words in calibration data and to calculate their frequency of use. The detection device may be programmed to recognize words in input data and to calculate their frequency of use. The detection device may be programmed to compare the calculated frequency of use of words associated with emotions of the input data to the calculated frequency of use in the calibration data and beyond.

Preferably, the processing unit comprises a deep neural network, wherein the processing unit is adapted to embed the extracted emotional features of the input data and the calibration data into the deep neural network and to create an emotional landscape of the user.

This allows for an easy and quick analysis of input data with a more precise reading of the displayed emotion.

‘Emotional landscape’ here refers to a higher dimensional vector space, so a collection of calibration data feature representations together. All input data may pass through the deep network and may be transformed to a high dimensional feature or vector. As the input data may also be incorporated in the deep neural network, this allows for the deep neural network to generate a more detailed emotional landscape the more the user uses the detection device.

In particular, the processing unit may be programmed to embed the calibration data and/or the processed data in a deep neural network. The processing unit may be programmed to create an emotional landscape of the user by using the calibration data and/or the processed data, especially extracted emotional features of the user. The processing unit may be programmed to calculate reference data points for emotions, especially feature vectors.

Especially, the processing unit may be programmed to compare said input data to said calibration data, especially to reference data points, especially by extracting emotional features and/or feature vectors. Especially, the processing unit is programmed to embed the input data and/or the extracted emotional features and/or the feature vectors into the emotional landscape and to calculate the nearest approximation of the input data and/or emotional feature and/or feature vector to reference data in the emotional landscape and/or to reference data points. The processing unit may be programmed to tag input data as a certain emotion based on the calculation.

The nearest approximation allows for an easy and quick detection of an emotion the user displayed in the time the input data was recorded.

The processing unit may comprise a computer program structure for analyzing input data of the user, especially video and/or audio data. The computer program structure may comprise an analysis mode. In the analysis mode, the processing unit may be programmed to gather input data of the user and/or send instructions to record themselves to the user. In the analysis mode, the processing unit may be programmed to prepare the input data for analysis, to embed the input data or the prepared data into a deep neural network and to calculate the similarities and differences between the input data and/or the processed data to the reference data.

Preferably, the detection device is adapted to analyze multiple input data over a period of time and is adapted to calculate an emotional state of a user based on said analysis.

If, over a period of time (the period of time may be predefined such as a month, two weeks etc.), the emotions detected by the detection device are more negative, this may indicate a negative emotional state. If the emotions detected are more positive, this may indicate a positive emotional state. This allows for an objective estimation of a positive or negative mindset of the user.

Additionally or alternatively, the detection device may be programmed to calculate a tendency of negative or positive emotions based on the analysis of multiple input data. Additionally or alternatively, the detection device may be programmed to calculate a risk of a mental illness and/or diagnose a mental illness.

The detection device may be adapted to provide exercise instructions for counteracting calculated negative emotions and/or for treatment of the diagnosed mental illness.

The exercise instructions provided for may be part of at least one of
breathing exercises,
journaling,
meditation,
Cognitive behavioral exercises,
Physical exercises,
etc.

The detection device may be adapted to provide alternative exercises based on further input data recorded by the user and/or based on user feedback. The detection device may be adapted to analyze exercise data of the user, which is recorded during and/or after the exercise. The detection device may be adapted to provide further exercise instructions and/or different exercises based on said analysis of exercise data.

To be adapted to analyze multiple data over a period of time, the detection device provides an emotional landscape, where the input data is stored for mapping an emotional state of the user. The input data may be tagged with a time and/or date tag. The detection device may be adapted to calculate and display an emotion curve over time. The detection device may be adapted to calculate times and/or places, when and/or where the user showed more positive or negative emotions.

Preferably, the detection device comprises a self-supervised learning computer program structure for learning to extract meaningful features of data of a user for emotion detection. The self-supervised learning computer program structure is adapted to learn to predict matching audio and video data of a user.

This allows for the program to use less data intense learning of relevant emotional features. In the state of the arts, a self-supervised learning program structure needs a lot of data to learn relevant emotional features. The above mentioned self-supervised learning computer program structure allows for a quick and less data intense process.

The detection device may comprise a Siamese neural network for self-supervised audio-visual feature learning. The Siamese neural network may be adapted to be fed a first batch of sets of audio and video data of multiple subjects, wherein the audio and video data would be separated from each other and to predict a correlation between said audio data and said video data of the first batch.

The Siamese neural network may be adapted to repeat the process with a second batch. Especially, the Siamese neural network may be adapted to repeat the process with a second batch of multiple, in particular five, sets of matching audio and video data of a singular subject, which are separated before being fed into the Siamese neural network. Especially, the second batch of sets of video and audio data concern the user in different emotional states.

The detection apparatus may comprise a deep network trained for analyzing video and/or audio data. The deep network may be stored at least partially in the main data storage.

Additionally or alternatively, the self-supervised learning computer program structure may be programmed to predict the calibration data to disclose certain emotions based on pretraining based on emotions data banks and to learn from the tagging of the calibration data as a certain emotion by the user.

The detection device may be programmed to instruct a user to consult a doctor or therapist for diagnosis and/or treatment of a mental illness.

Alternatively or additionally, the detection device may be programmed to indicate a probability of a mental illness and/or diagnose a mental illness based on a series of analysis of the emotional state of the user.

The detection device may be adapted to create instructions for the user to counteract negative emotions, such as instructions for breathing exercises, meditation, journaling, physical exercises, cognitive behavioral exercises.

The detection device may be programmed to predict the display of an emotion of input data recorded in a certain location and/or at a certain time. The detection device may comprise a self-supervised learning computer program structure for learning to extract meaningful features of data of a user for emotion detection, the self-supervised learning computer program structure being adapted to predict audio and video data relations, and/or relations of emotional features to time and/or location and/or situation recorded. The detection device may be programmed to send the user instructions to counteract negative emotions, if a certain time or location approaches.

Especially, the detection device may comprise one or both of the following:
a computer program structure for extracting feature vectors regarding the display of emotion of user data,
a self-supervised learning computer program structure for learning audio-visual features by associating interrelated video and audio data.

The self-supervised learning computer program structure may be pretrained. The self-supervised learning computer program structure may comprise commands to follow a pretraining method; the method may comprise the steps of
Mixing audio data and video data,
Extracting facial features of every video data and vocal features of every audio data,
Embedding the audio and video data into the network,
Signaling to learn features based on matching audio and video data.

The advantages over the state of the arts are described below in regards to the method for detecting the emotional state of the user.

The computer program structure or the self-supervised learning computer program structure may comprise commands for executing an analysis method. Additionally or alternatively, the processing unit may be adapted to perform at least one of the following steps of the analysis method:
associating and/or predicting an association of video and audio data of an emotions databank,
starting a computer-guided calibration sequence upon receiving a user instruction to calibrate via the connection element,
sending the user the instruction to record themselves via the connection element, the instruction comprising written or spoken notes on the content of the necessary recordings, such as notes on the type of recording device to be used, in this case a microphone and a camera, and directions on what the recordings should comprise, such as the direction to recording their face and voice while recounting the events of the present or remembering events evoking certain emotions,
storing, in particular temporarily, the data transmitted by the user via the connection element as calibration data,
sending the user the instruction to tag the calibration data with an emotions tag via the connection element and/or tagging the calibration data as a certain emotion,
processing calibration data, especially by embedding calibration data into a deep neural network, in particular processing calibration data into reference data by extracting feature vectors,
creating an emotional landscape of the user based on the calibration data and/or reference data and/or an emotions database,
sending the user instructions to record themselves in regular intervals such as days and/or recording the user in regular intervals such as days, as input data to be analyzed,
processing input data, in particular processing input data by extracting feature vectors of the input data, and by embedding the input data into an, especially calibrated to the user, deep neural network, especially into the emotional landscape of the user,
comparing input data to calibration data and/or reference data, especially comparing the feature vectors of input data to the feature vectors of calibration data and/or reference data,
finding the closest match between the input data and the reference data and/or finding a closely related reference point of the reference data,
calculating a distance of feature vectors of the input data to the feature vectors of calibration data and/or reference data,
denoting the input data as displaying a certain emotion,
calculating a tendency of displaying negative and/or positive emotions based on the analysis of multiple input data,
indicate the risk and/or probability and/or presence of a mental illness,
instruct the user to consult a doctor or therapist for diagnosis and/or treatment of a mental illness,
instructing the user to counteract negative emotions and/or treat the mental illness, especially instructing the user to follow exercises, such as breathing exercises, meditation, journaling, physical exercises, cognitive behavioral exercises,
instructing the user to record themselves during or after the exercises and analyzing the data recorded for improvements, especially adapting the instructions for exercises with no calculated improvements, in particular over no calculated improvements over a certain amount of time.

Preferably at least one of the main data storage unit and an additional temporary storage unit is adapted for temporarily storing the input data. Preferably, the main data storage unit or the temporary storage unit is adapted to store input data until the processing unit processed, and in particular analyzed, the input data and has outputted the calculated emotion or emotional state of the user.

This allows for better data security. Processed data is not quickly and easily identifiable as belonging to the user. Input data may contain private information, which is not recognizable after the input data is processed.

In particular, the main data storage unit or the temporary data storage unit may be adapted to delete and/or overwrite input data after storing the processed data and/or after a certain time has passed.

Preferably, the connection device comprises a wired and/or wireless connection element for communication with an interface device. In particular the connection element is a Bluetooth element.

This allows for an easy transfer of data. Especially, the connection device comprises a wired connection element for connecting the detection device to an interface device and a wireless connection element for connecting to a recording device.

The problems are also solved by a system for detecting the emotional state of a user. The system comprises a detection device according to any one of the preceding claims and at least one recording device for sensing the user, especially at least one of a camera and a microphone.

This system allows for an easy detection of the emotional state of a user.

The recording device may be adapted to sense the user. Especially, the recording device may be adapted to sense calibration data of the user and/or input data of the user. The system may be adapted to

The system may be adapted to automatically record the user, especially when the user uses the recording device for phone calls, video calls etc., in particular record the user periodically, such as daily, for a certain amount of time, such as less than 30 seconds, and/or the system may be adapted to inform the user, in particular periodically, such as daily, that recordings should be made. The system may be programmed to automatically update input data gathered by the recording device, when the recording device is connected to the detection device by the connection element.

The system may comprise multiple of the same kind of sensor, multiple microphones, multiple cameras etc. Especially, the system may comprise a microphone for user sensing and a microphone for environment sensing. This would allow for an easier and quicker way to denoising data of the user.

Preferably, the system is adapted to be calibrated to the user by use of the processing unit and calibration data recorded by the recording device.

Such a system allows for an easy and quick calibration to a certain user. In particular, the system may be programmed to be calibrated to the user by use of the at least one recording device and the detection device. Especially, the system comprises at least two recording devices, such as a camera and a microphone.

Preferably, the system comprises an ear-piece, wherein the recording device is part of this ear-piece.

This arrangement allows for a better quality of data of the user, as the recording device is closer to the user. Further, this ear-piece is easily transportable in day-to-day life.

Preferably, the ear-piece comprises a speaker and/or one or more additional recording devices, in particular an acceleration sensor and/or a photoplethysmographic sensor and/or a galvanic skin response sensor and/or an electroencephalogram sensor.

If the ear piece has a speaker, this allows the system to be used easily in day-to-day life. The ear-piece may comprise a communication element for communicating with an interface device, such as a cell phone. This allows easy recording of input data during a phone call or video call.

Preferably, the system comprises an interface device for interaction between the system and the user, in particular a smartphone or tablet, the interface device being connected to the detection device by the connecting element.

This allows for an easy and quick calibration of the system to the user and communication between the system and the user.

The interface device may comprise a display element for displaying information, particularly at least one of instruction information and analyzation results. The interface device may comprise an interacting element adapted to allow interactions with a user. The interacting element and the display element may be combined into a communication device, such as a touch screen. The interface device may comprise one or more recording devices, such as a camera, a microphone, an acceleration sensor, a location sensor, such as GPS, etc.

The interface device may comprise a computer program structure allowing an interaction between the interface device and the detection device. The detection device may record the data and may send it entirely or pre-processed to the app of the interface device, using wireless or wired connections. The interface may be programmed to further process the data and comparing the results to other users. The interface device may show the user the measured data and may be programmed to learn from the data of other users and may be programmed to give advice to the user such as: “You laughed less than 10% of the average. Try to laugh more” or “Your voice sounds worried compared to your normal state, what's going on?”.

In particular, the system may be adapted to be set in a calibration mode upon instructions entered by the user in the interface device. The system may be adapted to present the user with instructions on the use of the recording device for calibration of the detection device, on the interface device and/or via the speakers of the ear-piece and/or via speakers of the interface device. The system may be adapted to guide the user through calibration by displaying calibration instructions on the interface device and/or via the speakers of the ear-piece and/or via speakers of the interface device. The calibration instructions may comprise at least one of the following instructions: recording instructions, instructions of remembering a day or event, especially a day or event provoking a certain emotion, instructions to tag the recorded data with an emotions tag. The interface device, especially the interacting element, may be adapted to allow the user to start the calibration of the system to the user. The system may be adapted to record the user with the at least one recording device, especially upon entry of recording instructions by the user.

Especially, the interface device may comprise an interface storage unit and an interface processing unit.

The system may be adapted to indicate interferences with the recognition of emotional features such as high background noise levels or low light levels and to indicate, when such interferences are adjusted appropriately.

The detection device may be adapted to matching input data of the user recorded by the at least one recording device to reference data recorded by the at least one recording device.

The interface device may be adapted to allow user interaction with at least one of the processing unit, the at least one recording device and/or the main storage unit.

The system may be programmed to follow a calibration process; the calibration process may comprise any of the steps of:
presenting recording instructions to the user via the interface device and/or ear-piece for a first part of the calibration process, the recording instructions comprising instructions to the user of the use of a or multiple recording devices, especially of using a camera and a microphone, and instructing the user to recount the present or past day,
recording the user via the recording device or devices for the first part of the calibration process, especially on input of the user via the interface device,
presenting recording instructions to the user via the interface device and/or the ear-piece for a second part of the calibration process, the recording instructions comprising instructions to the user of the use of a or multiple recording devices, especially of using a camera and a microphone, and instructing the user to recount one memory of an event provoking a certain emotion, and to repeat this with other memories, each memory provoking a certain emotion,
recording the user via the recording device or devices for the second part of the calibration process, especially on input of the user via the interface device,
tagging the recorded calibration data by the user via the interface device as displaying a certain emotion,
extracting emotional features such as described above, especially feature vectors,
embedding the tagged calibration data and/or the extracted emotional features and/or feature vectors in the deep neural network
creating an emotional landscape of the user.

Such a system allows for a more precise calibration to a certain user while using a moderate amount of records of the user, thus presenting a low burden on the user.

Preferably, the system comprises multiple recording devices. The recording devices comprise an acceleration sensor and/or a temperature sensor and/or a humidity sensor.

The sensors allow for the gathering of more calibration data and/or input data of the user. With this information, the calibration and/or analysis and thus the calculated emotions and emotional state are more precise. Stress, fear and other emotions have a direct impact on the whole human body, such that the above mentioned sensors provide insight in the emotions felt by the user.

The problems are also solved by a computer-implemented method for detecting an emotional state of a user, comprising the steps of
Providing a detection device, in particular a detection device as previously described, and/or a system, in particular a system as previously described, a recording device, in particular a camera and/or a microphone, preferably at least one camera and at least one microphone, and an interface device, in particular a smartphone or tablet,
Recording calibration data of the user by use of the recording device, wherein particularly the calibration data is audio data of the user and/or video data of the user, in particular at least one, preferably multiple, sets of audio and video data, and in particular temporarily storing the calibration data as input information in the main storage unit or the temporary storing unit,
Calibrating the detection device to the user by processing this calibration data by a processing unit of the detection device, especially embedding the calibration data into a trained deep neural network,
Recording input data by the recording device, wherein particularly the input data is audio data and/or video data of the user,
Analysing the input data by the processing unit by comparing input data to the reference data,
Determining a commonality of the input data and reference data, in particular identifying an emotion,
In particular outputting the determination on the interface device.

This method allows for an easy, quick and precise detection of the display of an emotion of a user.

Usually, emotion detection relies merely on computer-learning of a vast amount of data of different people, without a specific calibration of the device to the user. The use of sets of matching video and audio data has the advantage of allowing a more precise calibration.

The method may comprise steps of calculating feature vectors of calibration data and input data, and calculating the closest match of the feature vectors of the input data and the feature vectors of the calibration data. Especially, the method may comprise the step of calculating a distance of the feature vectors of the input data to the feature vectors of the calibration data. This allows for the assessment of certainty of the precision of the calculated emotion.

Especially, the method may comprise any one of the following steps:
Creating an emotional landscape of the user,
Embedding input data into the emotional landscape of the user,
Creating data reference points for certain emotions in the emotional landscape,
Presenting calibration instructions of a first part of a calibration process to the user via the interface device, especially instructions to remember general memories not specifically related to a certain emotion,
Recording a first set of calibration data of the user by the at least one recording device, especially by a microphone and a camera,
Presenting calibration instructions of a second part of the calibration process to the user via the interface device, especially instructions to remember events specifically related to a certain emotion,
Recording a second set of calibration data of the user by the at least one recording device, especially by a microphone and a camera,
Tagging the calibration data by the user via the interface device with an emotions tag.

The method may comprise the steps of pretraining of the self-supervised audio-visual feature learning structure, especially a Siamese neural network.

In particular, this pretraining comprises the steps of:
Feeding in matching audio and video data of multiple subjects, in particular five subjects,
Mixing audio data and video data,
Extracting facial features of every video data and vocal features of every audio data,
Embedding the audio and video data into the network,
Signaling to learn features based on matching audio and video data.

In the state of the art, the method for a self-supervised structure to learn features is by analyzing the data of large datasets. For audio-visual features, there are three types of datasets: natural datasets, semi-natural datasets and simulated datasets.

Natural datasets are extracted from videos and audios recordings for example those available on online platforms. Databases from call centers and similar environments also exist such as VAM, AIBO, and call center data. Modeling and detection of the emotions with this type of datasets can be complicated due to the continuousness of emotions and their dynamic variation during the course of the speech, and the existence of concurrent emotions. Also, the recordings often suffer from the presence of background noise which reduces the quality of the data.

Semi-natural datasets are relying on professional voice actors playing defined scenarios. Examples of such datasets are IEMOCAP, Belfast, and NIMITEK. This type of datasets includes utterances of speech very similar to the natural type above but the resulting emotions remain artificially created, especially when speakers know that they are being recorded for analysis reasons. Additionally, due to the limitations of the situations in scenarios, they have a limited number of emotions.

The simulated datasets also rely on actors but this time they are acting the same sentences with different emotions. Simulated datasets are EMO-DB (German), DES (Danish), RAVDESS, TESS, and CREMA-D. This type of datasets tends to have overfitted models around emotions slightly different than what is happening in day-to-day conversations.

Thus, each type of dataset has its own drawbacks. The audio-visual learning progress in each case, however, requires a large amount of data processing. Thus, the proposed method for pretraining the self-supervised learning structure has the unique advantage of needing less data processing than the known state of the art. Further, to reach a similar learning process to adapt the devices in the state of the arts to the user, tens of thousands of records of the user would need to be taken. A pretrained self-supervised audio-visual feature learning structure as described above, uses far less data of the user and thus presents less of a burden to the user in the calibration process, and still presents a precise calibration to a specific user.

Preferably, the method comprises the following steps:
preparing calibration data
preparing input data,
   wherein the calibration data and the input data is video data of the user and/or audio data of the user, wherein the preparing includes at least one of
Extracting bounding boxes to detect frontal faces, in particular in every video frame, and
Cropping video data, in particular the face bounding boxes, and
Resizing video data, especially the face bounding boxes, and
Splitting audio data into time windows of a certain length, in particular time windows of less than 5 seconds, and
Splitting audio data, in particular time windows of said audio data, into frames.

This allows for a quicker and more precise analysis of raw data.

The method may comprise the step of splitting video data into time windows of a certain length, in particular into time windows smaller than 5 seconds, especially the time windows are 1.2 seconds. The method may comprise the step of showing the video data time windows of the calibration data to the user for tagging it as a certain emotion.

Preferably, the method comprises the steps of
Extracting emotional features of the calibration data, in particular prepared calibration data,
Extracting emotional features of the input data, in particular prepared input data,
   wherein an emotional feature extracted is at least one of
A fundamental frequency of a voice
A Formant frequency of a voice
Jitter of a voice
Shimmer of a voice
Intensity of a voice
Pixels, especially pixels regarding facial expressions or posture.

More features may include:
Detect number of laughs, coughs, clearing throat, ‘ems’, etc. from voice.
Blinking rate, pupil size, eye movements including saccades from video
Step detection, tremor detection, gait analysis, body posture estimation using accelerometers
Typing time stamps for each character from the keyboard in order to detect fatigue, concentration problems etc. of the typer.
Extracting humidity of the skin on the ear using sensors in the head phone.
Extracting air humidity and detect effect of environmental humidity on the emotional state of the user.
Extracting respiratory rate using microphone in the hearables
Using EKG data from fitness monitor or hearables or other device including at least one parameter of.
Heart rate
   [symbol] Heart rate variability
   [symbol] Ischemia
   [symbol] Heart attack

These features have been shown to allow a precise analysis of the users' emotions as mentioned above.

The method may comprise the step of combining the extracted emotional features into a data reference point for a certain emotion.

Preferably, the method comprises the steps of
Recording one or multiple, in particular at least five, input data of the user,
Determining an emotional state of the user, in particular by calculating a tendency of negative or positive emotions,
Outputting the determined emotional state on the interface device.

This allows for an easy analysis of the emotional state of the user.

The recording may be periodically, as in daily. The input data may be recorded over multiple days, such as one recording a day for six days, and/or the input data may be recorded multiple times on a single day, such as twice a day or twice a day for three days. The record data may be recorded during a phone call the user marked as to be recorded and/or by voice recording the user journaling.

Additionally or alternatively, the method may comprise at least one of the steps:
Calculating a tendency of negative or positive emotions,
Calculating a risk for a mental illness,
Diagnosing a mental illness,
Outputting the calculations and/or diagnoses on the interface device,
Generating exercises based on calculated emotions, the determined emotional state and/or calculated risk for a mental illness and/or diagnosed mental illness,
Providing exercise instructions on the interface device and/or through the ear-piece.

The problems are also solved by a computer program product comprising instructions which, when the program is executed by a computer, especially a detection device as previously described and/or a system as previously described, cause the computer to carry out the steps of a method for detecting the emotional state of the user.

The computer program product may comprise a self-supervised learning structure for learning to extract meaningful features for emotion detection. The self-supervised learning structure may be adapted to, when fed with multiple pairs of matching video and audio data, learn to predict, which video and audio correspond. The self-supervised learning structure may also be adapted to learn to filter between relevant data and irrelevant data.

In particular, the self-supervised learning structure may comprise commands to follow a computer-implemented method for self-supervised audio-visual feature learning as described below.

The self-supervised learning computer program structure may comprise a proxy task structure for mixing multiple, in particular 5, sets of video and audio data, especially of different subjects, and predicting, which video and audio match.

Thus, when fed with multiple matching video and audio data, especially video data concerning faces and audio data concerning voices, the software product learns to correlate the video data and the audio date, especially learns to match people's facial expressions and their tonality.

The problem is also solved by a computer-implemented method for self-supervised audio-visual feature learning; the method comprises the steps of
Providing a Siamese neural network
Feeding in a first batch of sets of matching audio and video data of multiple subjects into the Siamese neural network, especially between 3-5 subjects,
Separating the audio data from the video data of the first batch,
Mixing the audio data and the video data of the first batch,
Predicting a correlation between said audio data and said video data of the first batch,
Feeding in a second batch of sets of matching video and audio data, especially of a singular subject, in particular 5 sets of matching audio and video data of this subject,
Separating the audio data from the video data of the second batch,
Mixing the audio data and the video data of the second batch,
Predicting a correlation between said audio data and said video data of the second batch.

Such a method allows training a self-supervised learning computer program structure to be more robust to changes in microphone, video camera, position of the camera and background noise. Further, this method leads to less dependence on the specification of devices and allows the Siamese neural network to be used across cultures. Even more, this method allows for training to recognize facial micro-expressions and voice tonality. The use of a Siamese neural network ensures that each audio and video clip is embedded in their respective representation space.

In particular, more subjects can be used, such as 100-100000 subjects.

The method may comprise the steps of providing a self-supervised learning computer program structure as previously described and/or any one or more step of pretraining as previously described.

The method may comprise the step of providing a classification task of learning to predict a correspondence between audio data of voices and video data of faces.

The method may comprise the steps of extracting audio features of the voice of the audio data, extracting video features of the face of the video data.

The method may comprise any one or any combination of the following steps:
instructing a user to remember an event and/or day,
recording the user recounting the event and/or day, as a third batch of sets of audio and video data,
feeding a third batch of sets of audio and video data of a user into the Siamese neural network,
predicting the sets to be tagged with a certain emotion,
tagging the sets of audio and video data of the third batch as a certain emotion, in particular by the user,
instructing the user to remember an event and/or day, in particular remembering an event and/or day connected to a certain emotion,
recording the user recounting a certain event and/or day connected to a certain emotion, as a fourth batch of sets of audio and video data,
feeding a fourth batch of sets of audio and video data of a user into the Siamese neural network, in particular the audio and video data being automatically tagged with the emotions tag relating to the emotion the event or day was instructed to provoke,
embedding a third and/or fourth batch of video and audio data into the Siamese neural network,
creating an emotional landscape based on the embedding of the third and/or fourth batch.

This method allows the Siamese neural network to learn to adapt do a certain user.

The problem is also solved by a trained machine-learning model, trained in accordance with the computer-implemented method for self-supervised audio-visual feature learning as previously described.

The problems are further solved by a computer program product comprising instructions which, when the program is executed by a computer, cause the computer to carry out the steps of the method as previously described.

The computer program product may comprise a self-supervised learning computer program structure, especially a self-supervised learning computer program structure as previously described.

The computer program product may comprise pretrained face-recognition structures to extract features from video frames.

Differently than other known detection devices, the detection device of this invention presents a way for analyzing recorded data despite poor quality of the raw data due to distance to the subject, background noise and other interferences.

The computer program product may comprise a calibration setting, wherein the calibration setting allows for at least one of giving instructions to a user concerning the calibration and processing calibration data into reference data. In particular, when the software product is set to calibration, the software product sends instruction information to a display device for the user to follow. In particular, the user may be instructed to record themselves with at least one recording device, preferably with two recording devices, especially with a microphone and a camera. In particular, the user may be instructed to record themselves narrating their day or events in their life, which in particular concern different emotional states. The recording device or devices record said user, in particular in multiple instances. Further, the user may be instructed to tag the recorded data as displaying a certain emotion the user identifies in the recorded data.

The recorded data may be stored as calibration data, in particular temporarily, on at least one of a main storage unit or a temporary storage unit. In the calibration setting, the software product may be adapted to process said calibration data into reference data.

The software product comprises an analysing setting, wherein the analysing setting allows for analysing data recorded by a recording device by comparing data to be analysed to reference data.

The software product is adapted to analyse recordings of recording devices of different quality, by use of computer based learning.

This allows for a user to simply record themselves without having to prepare a certain stage and consider background, noise or other interferences.

The problems are also solved by a method for detecting an emotional state of a user carried out by a computer, in particular a detection device as previously described or a system as previously described, the method comprising at least one or any combination of the following steps:
Providing a computer, especially a detection device, trained by the computer-implemented method for self-supervised audio-visual feature learning as previously described,
instructing a user to record themselves in different emotional states and/or instructing a user to record themselves remembering events evoking certain emotions, as calibration data,
instructing the user to tagging the calibration data as a certain emotion and/or tagging the calibration data as a certain emotion,
processing calibration data, especially embedding calibration data into a deep neural network, particularly processing calibration data into reference data,
extracting emotional features such as mentioned above of input data and/or calibration data, especially calculating feature vectors
creating an emotional landscape of the user based on the calibration data and/or an emotions database,
instructing a user to record themselves and/or recording the user, as input data, especially daily and in particular daily for at least a week,
embedding the input data into the deep neural network, especially into the emotional landscape of the user,
calculating a similarity between feature vectors of input data and feature vectors of calibration data,
calculating a distance between feature vectors of input data to feature vectors of calibration data,
comparing the input data to reference data,
finding the closest match between the input data and the reference data and/or finding a closely related reference point of the reference data,
denoting the input data as displaying a certain emotion,
calculating a tendency of negative or positive emotions based on the analysis of multiple input data,
outputting the calculation results,
indicating the probability and/or presence of a mental illness,
instruct the user to consult a doctor or therapist for diagnosis and/or treatment of a mental illness,
instructing the user to counteract negative emotions and/or treat the mental illness, especially, instructing the user to follow exercises, such as breathing exercises, meditation, journaling, physical exercises, cognitive behavioral exercises,
adjusting exercise instructions to the user based on user feedback, especially on time constraints,
instructing the user to record themselves during or after the exercises and analyzing the data recorded for improvements, especially adapting the instructions for exercises with no calculated improvements, in particular over no calculated improvements over a certain amount of time.

This method allows for a quick and easy way of detecting an emotion displayed by a user and/or their emotional state and/or a risk of a mental illness and/or a diagnosis of a mental illness Especially, this method provides the user a unique and unbiased look into their emotional state and instructions to improve their emotional state which are adaptable to a busy lifestyle.

The method may comprise other steps as described for a method for detecting the emotional state of a user as previously described.

The problems are also solved by a detection device for detecting the emotional state of the user. The detection device is trained by the computer-implemented method for self-supervised audio-visual feature learning and/or the detection device comprises means to carry out a computer-implemented method for self-supervised audio-visual feature learning as previously described and/or the detection device comprises means for carrying out a method for detecting an emotional state of a user as previously described.

This detection device provides precise readings on the emotional state of the user, as it is adapted to the user.

Further, the detection device may comprise other elements of a detection device for detecting the emotional state of a user as previously described, in particular a processing unit as previously described, a main data storage unit as previously described and/or a connection element as previously described.

The problem is also solved by a system comprising a detection device as previously described, at least one recording device as previously described and an interface device as previously described.

The problems are also solved by a computer program comprising instructions which, when the program is executed by a computer, especially a detection device as previously described or a system as previously described, cause the computer to carry out at least one of the steps of a computer-implemented method for self-supervised audio-visual feature learning as previously described and/or a method for detecting an emotional state of a user as previously described.

A computer-readable medium, especially a main data storage unit, in particular a main data storage unit as previously described, comprising instructions which, when executed by a computer, such as a detection device as previously described or a system as previously described, cause the computer to carry out a computer-implemented method for self-supervised audio-visual feature learning as previously described and/or a method for detecting an emotional state of a user as previously described.

### BRIEF DESCRIPTION OF THE DRAWINGS

The invention will be further outlined in the following with reference to preferred embodiments with drawings, without being limited thereto. The figures show:

FIG. 1 a representation of a detection device

FIG. 2 a diagram of a detection system

FIG. 3 a diagram of a calibration process

FIG. 4 a diagram of a computer-implemented method for recognizing an emotional state of a user

FIG. 5 a diagram of the interaction of the software of the detection system of FIG. 2

FIG. 6 a diagram of an analysis process of data clips

FIG. 7 a diagram of a self-supervised audio-visual feature learning process

FIG. 8 a diagram of a computer program interworking

### DETAILED DESCRIPTION OF THE PREFERRED EMBODIMENTS

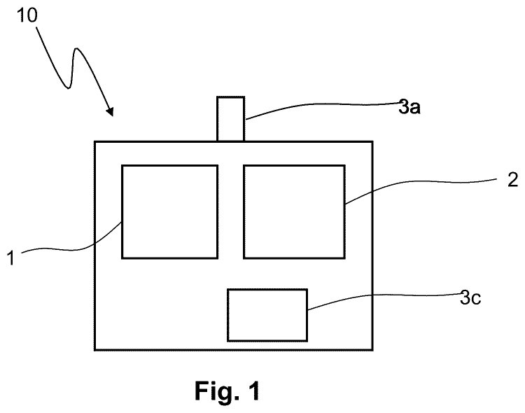

FIG. 1 discloses a representation of a detection device 10 for detecting the emotional state of a user. The detection device 10 comprises a processing unit 1, a main data storage unit 2, a wired connecting element 3a for connecting with an interface device and a wireless connection element 3c for connecting with a recording device.

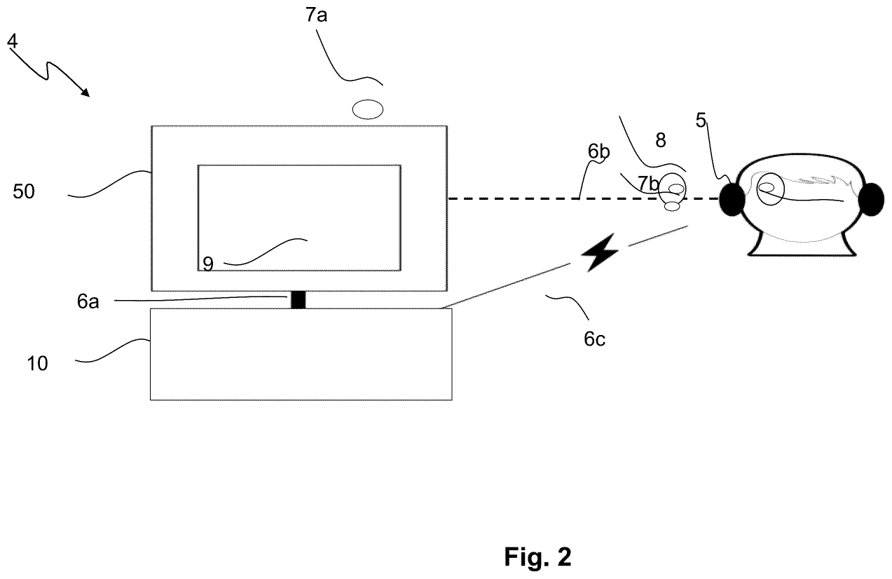

The processing unit 1 is adapted for processing data, in this case calibration data and input data. The main data storage unit 2 comprises a storage capacity of at least 1 GB and is used to store calibration and input data, at least until prepared to be processed by the processing unit 1. The connecting element 3c is a Bluetooth element for connecting to a recording device (see FIG. 2.)

Calibration data is data of a specific user, such as their voice, face, posture and other data of the users body, recorded by a recording device (see FIG. 2), and used for calibrating the detection device to the user in a calibration mode of the detection device 10. Calibration data is tagged by the user with an emotions tag, denoting a certain recording of the user as a certain emotion. Such an emotion may be “tired”, “happy”, “relaxed”, “fear”, “alarmed”, “excited”, “astonished”, “delighted”, “pleased”, “content”, “serene”, “calm”, “sleepy”, “bored”, “depressed”, “miserable”, “frustrated”, “annoyed”, “angry”, “afraid”, “neutral”, etc.

Input data is also data of the same specific user, such as their voice, face, posture and other data of the users body recorded by a recording device (see FIG. 2), and is used for determining the emotional state of the user in the analysis mode.

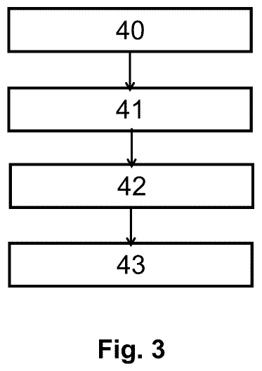

The process for detecting the emotional state of the user begins by calibrating the detection device 10 to the user. For this, the user connects the detection device 10 via the connecting element 3c to a source for raw recorded data, such as a recording device 7b (see FIG. 2). Recorded data of the user, in this case data recorded by a camera and a microphone, is entered into the main data storage 2. The recorded data is tagged by the user with an emotions tag, indicating that the tagged data discloses a certain emotion, such as happiness, sadness, anger etc. and used by the processing unit 1 as calibration data for calibrating the detection device. The process of calibration is further described in the description to FIG. 3.

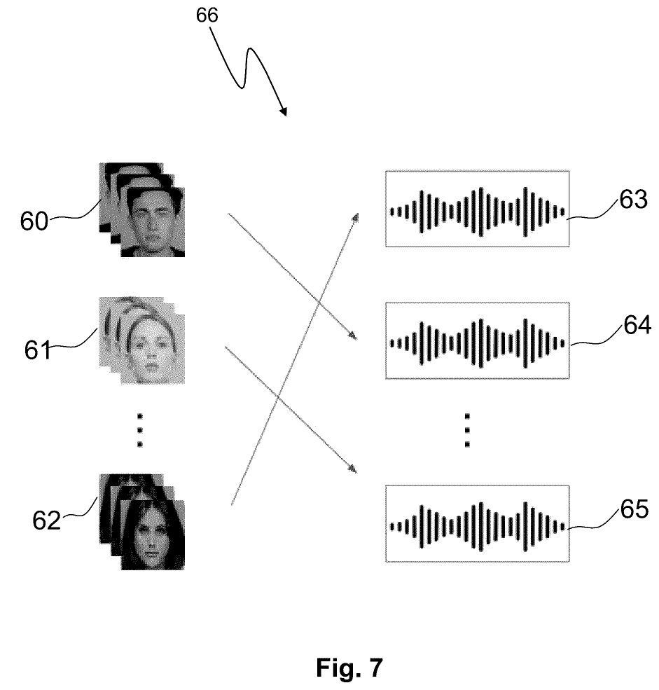

For the calibration and analysis, the detection device (10) comprises a trained deep neural network, in which the tagged calibration data is embedded for the calibration. The detection device (10) creates an emotional landscape of the user, providing reference points for certain emotions. Thus, when input data of the user is recorded and embedded into the landscape, the processing unit (1) can calculate the nearest approximation of the input data to reference points for certain emotions. The deep neural network comprises a self-supervised learning structure (66). The structure comprises a proxy task for n mix and match of several sets of video and audio clips, for learning to predict matches of audio clips to video clips. The details are explained in the description to FIG. 7.

For the analysis, the user then records further data sensed by a recording device, data recorded by a microphone or a camera, as audio or video clips. The detection device automatically transmits data to the interface device on a regular basis (as soon as there is connection or at least once per minute if connection is present). These audio or video clips are entered into the main data storage unit 2 as input data to be analysed by the detection device 10. The input data is compared to the calibration data and the input data is calculated as to show a certain emotion. The input data is only stored in the detection device 10 and, once processed, is deleted.

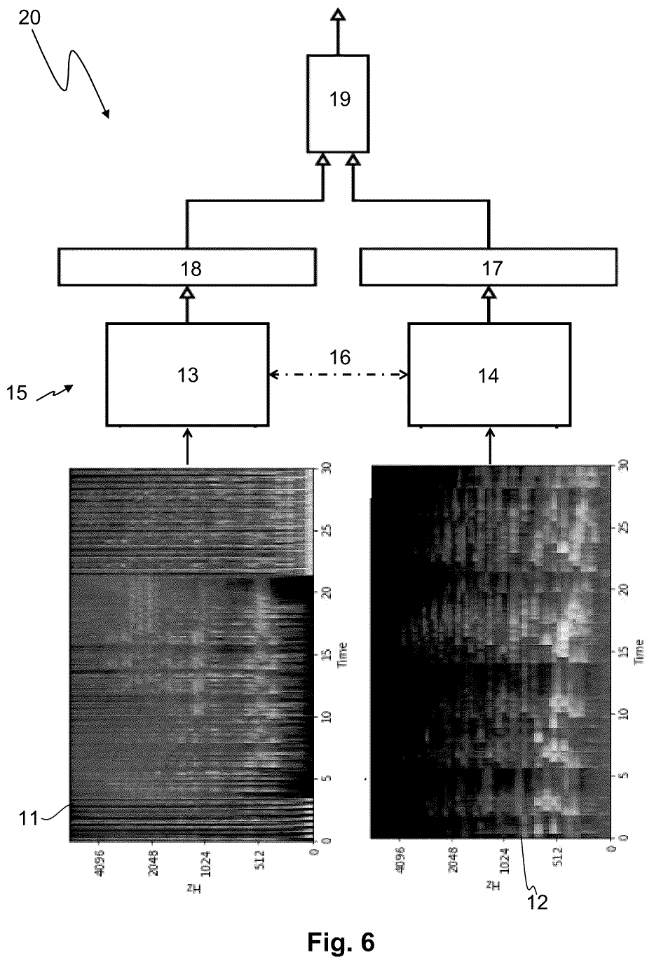

Multiple data clips are recorded, as in the input data is gathered over multiple days, in this case 10 second recordings of telephones, video calls or journaling per day. These input data clips are analysed to detect the emotion displayed in each of the data clips. The analysis method is further described in the description to FIG. 6. Based on the analysis of the multiple input data, the processing unit 1 calculates an emotional state of the user.

In case of the emotional state shown to be persistently negative, the processing unit 2 calculates a risk for and/or a diagnosis of a mental illness, such as depression, anxiety or schizophrenia.

The detection device 10 is programmed to send the calculation results, such as the calculated emotions, emotional state, risk for or diagnosis of a mental illness to an interface device (see FIG. 2).

FIG. 2 shows a representation of a system 4 for detecting an emotional state of a user. The system 4 comprises a detection device 10, an interface device 50 and an earpiece 5.

The interface device 50 is a smartphone or laptop, comprising a touch screen 9, or a screen and a key board. The interface device also comprises a first recording device 7a, in this case a camera. The camera 7a is adapted to film the users face. The ear-piece 5 comprises a speaker 8 and a second recording device 7b, in this case a microphone. The microphone 7b is adapted to sense the users voice.

The microphone in each ear-piece is composed of a number of pressure-sensitive sensors (typically 3 of them, of size smaller than 4 mm in diameter or edge, therefore very light weight and small) inserted in the earpiece around the ear facing the outside and/or at the entrance of the ear canal. The ear-piece comprises other openings to mount other sensors such as an acceleration sensor or an EEG sensor.

The detection device 10 is connected to the interface device 50 via a wired connection 6a and the ear-piece 5 can be connected to the interface device 50 and the detection device 10 via a Bluetooth connection 6a, 6c. The connections of the detection device 10 to the interface device 50 and the ear-piece 5 allows for sending and receiving data, especially for audio data and in case of the connection to the interface device 50 also for video data.

For the calibration, the camera 7a and the microphone 7b are used to record the user. The gathered data is sent as calibration data via the connections 6a and 6c to the detection device 10. After the calibration, input data audio clips are recorded by the microphone 7b, either during journaling and sent via the connection 6c to the detection device 10, or during a monitored phone/video-call using the interface device 50 and the ear-piece 10 and the audio and/or video clips are sent to the detection device 10 via the connections 6a and 6c. The input data clips are analysed by the detection device 10. The input data audio clips are deleted when the calculation of the emotion is completed. The detection device 10 sends the calculation results to the interface device 50, where they can be displayed on the touch screen 9. Further, exercise instructions are sent from the detection device 10 to the interface device 10 or to the ear-piece 5. The exercise instructions are either displayed on the touch screen 9 or played on the speaker 9. During the exercise, the user is recorded by the camera 7a and/or by the microphone 7b. The user can use the touch screen 9 to retrieve calculation results, to start the calibration process, to tag recorded data during the calibration with an emotions tag, to instruct the monitoring of a call and to adjust the exercise instructions. Based on the user input and the data recorded during the exercise, the instructions are adapted.

FIG. 3 shows a diagram of a calibration process for the detection device 10. In the calibration process, the detection device 10 (see FIGS. 1 and 2) is calibrated to the emotional display of a specific user:

In a first step 40, the user is instructed by the detection device 10 via the speakers 8 of the ear-piece or an app on the interface device 50 via the touch screen 9 to talk about their day while being recorded by two recording devices, a camera 7a and a microphone 7b (see FIG. 2). The camera 7a is a front facing camera like in a smart phone or laptop. The user then tags their audio-video clip based on the emotions they displayed in each segment with an emotions tag.

In a second step 41, the user again instructed via the means mentioned above (see also FIG. 2) to recount an event that provokes a certain emotion, such as an event that made them happy. This is repeated with different events provoking different emotions, such as sad, angry etc. For each emotion, 2-10 recounts of events are recorded. The user recounting those events is again being recorded by two recording devices, a camera 7a and a microphone 7b (see FIG. 2).

In a third step 42, the audio-video clips gathered in step 1 and 2 are embedded in a Siamese neural network 20 (see FIG. 6).

In a fourth step 43, the data embedded is prepared and emotional features of the clips are extracted.

This process allows the detection device to recognize, how the user expresses emotions. The two step approach of step 40 and step 41 allows for calibrating the detection device 10 with more versatility in the expressions of the user.

For the calibration of the detection device 10 with video and audio data, a pretrained self-supervised learning structure is used (see FIG. 7). This allows a calibration with less data of the specific user and thus for a quicker calibration.

As the audio-video clips gathered by the camera 7a and microphone 7b are not further prepared before being fed into the detection device 10, these audio-video clips are raw data. Before extracting emotional features of these clips, that is before extracting feature vectors, these clips need to be prepared.

The preparation and extraction of features of the video clips are as follows: First, bounding boxes of the video clip are extracted to detect frontal faces in every video frame. The face detector is based on an SSD face detector with a ResNet architecture. The face bounding boxes are further cropped and resized. To extract features from the face frames, a pretrained VGG-Face model is used. Pixels of the face are extracted as emotional features.

For the audio features, the recordings are split into time windows of size about 1.2 seconds. Each time window is itself split in frames of 25-ms with 10-ms overlap. Some features are evaluated per frame, while others require averaging over all the frames of a window. The features extracted include:
The fundamental frequency: The fundamental frequency changes with emotional arousal. The fundamental frequency parameter is the most affected parameter from the anxious state; this is shown in an increase in its value. The fundamental frequency is associated with the rate of glottic rotation and is considered a “prosody feature”, a feature that appears when sounds are put together in connected speech.
Jitter, shimmer, intensity parameters: Increases are observed in the anxious state and the sound intensity is irregular. In addition to anxiety, gender has an important influence on the jitter parameter and affects this parameter by 64.8%.
The parameters of the formant frequencies: Each formant is characterized by its own center frequency and bandwidth, and contains important information about the emotion. For example, people cannot produce vowels under stress and depression, and the same is true in the case of neutral feelings. This change in voice causes differences in formant bandwidths. The anxious state caused changes in formant frequencies. That is, in the case of anxiety, the vocalization of the vowels decreases. Specifically, the bandwidth of the formant frequencies, mel-frequency cepstral coefficients (MFCC), linear prediction cepstral coefficients (LPCC) and wavelet coefficients are affected by anxiety. A significant increase is observed especially in the wavelet coefficients.

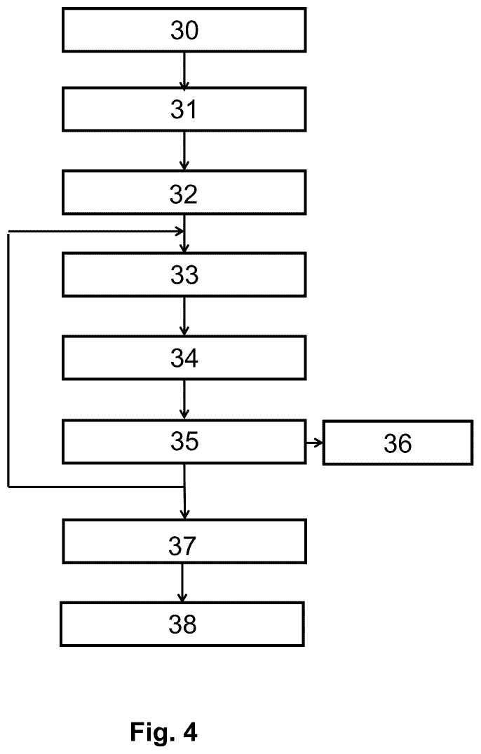

FIG. 4 shows a diagram of a computer-implemented method for recognizing an emotional state of a user.

In a first step 30, a system 4 (see FIG. 2) is provided.

In a second step 31, the audio-video clips of the user are recorded by a camera 7a and a microphone 7b (see FIG. 2) as a set as calibration data. Using both audio and video data has proven to be more reliable than using either one on their own.

In a third step 32, the detection device 10 is calibrated to the user, by creating an emotional landscape of the user.

The details to steps 31 and 32 are explained in the description to FIG. 3.

In a fourth step 33, an audio clip is recorded by the microphone 7b as input data. In detecting user's emotions for the day, ca. 10 seconds of speech is recorded by the user, for example when they initiate a call that they wish to monitor, or from journaling. The audio clip is sent directly from the microphone 7b to the detection device 10 (see FIG. 2).

In a fifth step 34, the input data is analysed by the processing unit by comparing it to the embedded and tagged calibration data, which is further called reference data. For this, the processing unit is programmed to embed the input data into the emotional landscape of the user and to look for the nearest neighbour vectors, tagged in the calibration, in the emotional landscape.

In a sixth step 35, the detection device 10 (see FIG. 2) determines a percentage similarity of the input data to certain reference data points. The majority consensus from multiple of the nearest neighbours allows the detection device to identifying the input data as a certain emotion. The distance to the nearest reference data point allows for a measurement of how reliable the identification of the emotion is.

The emotion recognized can be shown to the user on the interface device 10 in a seventh step 36.

The steps 33, 34 and 35 are repeated daily for at least a week. Based on multiple iterations of the steps 33, 34 and 35, an emotional state of the user is calculated in an eight step 37.

In a ninth step 38, the system generates instructions for therapeutic exercises if a negative emotional state has been calculated, to counteract the negative emotions of the user.

The detection device 10 or the system 4 comprises an exercise database related to significant exercises to counteract a specific emotion, such as sadness. The detection device 10 compiles a set of exercises based on the emotions detected in the input data and presents the user with the exercise instructions to these compiled exercises. The instructions are presented for the user to follow as a treatment in the moment, such as a step-by-step instruction on breathing for a meditation or as general advice, such as advice on how to deal with stress by limiting alcohol and caffeine intake. The exercise instructions are presented as records of an instructor's voice, which can be heard using the interface device or the ear-piece. The general advice is presented as written statements displayed on the interface device. The database is accessible to the user, who can access the database using the interface device 50 and compile the exercises they want to use. The database provides background information on why a certain advice or exercise is useful. The user is provided with a feedback option to allow the user to adjust the exercise instructions if a certain exercise proves to be more or less helpful. The efficacy of the exercises is measured automatically using above-mentioned emotional features.

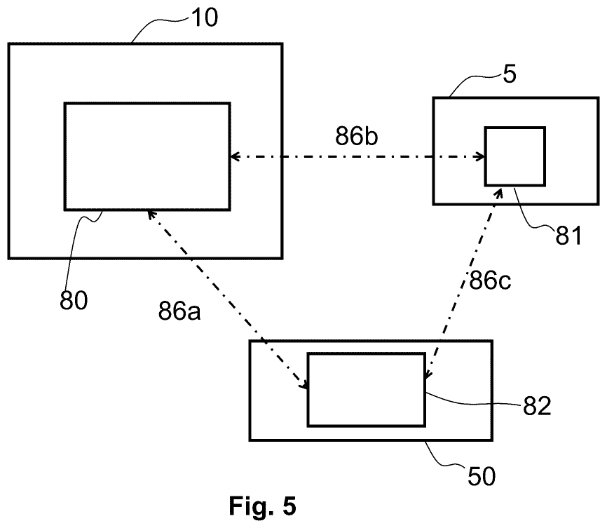

FIG. 5 shows a diagram of software of the system of FIG. 2. For the detection of the emotion of the user, the computer program product 80 of the detection device 10, the software 81 of the ear-piece 5 and the app 82 of the interface device 50 cooperate.

The computer program product 80 comprises a self-supervised audio-visual feature learning structure 66 (see FIG. 7) and a Siamese neural network 20 (see FIG. 6) for analysing the emotional display of the user.

The app 82 on the interface device 50 comprises functions for connecting to other devices and platforms, such as Apple health, Garmin, calendars, fitness apps etc., as well as of sensors of the interface device, such as cameras, GPS etc. This connection allows for analysing the circumstances of the user's emotions, like stressful meetings, locations, etc. The app 82 connects with the computer program product 80 to exchange data 86a, especially to send the data gathered from other devices, apps or sensors. Further, the app 82 comprises interaction functions, allowing the user to start calibration, tag input data as a certain emotion, request and adjust exercise instructions, adjust privacy settings, retrieve the calculation results and control other functions of the app 82, the computer program product 80 and the software 81 of the ear-piece. The app 82 allows for exchanging data 86c with the ear-piece and is programmed to monitor the audio data that is transmitted from the interface device 50 to the earpiece 5, which allows recording input data from phone calls the user wishes to monitor or analyse music consumption of the user as part of the determination of the emotional state of the user. The app 82 is also programmed to measure or use measures of other apps for monitoring sensor data such as heartrate or heartrate-variability before, during and after exercises instructed by the detection device 10.

The software 81 of the ear-piece 5 is programmed for recording calibration and input data by the microphone 7b and sending this data to the detection device 10.

The microphone sensors are combined to create a beam of sensitivity towards the mouth of the user so that clear speech may be retrieved and analyzed. As well, a comparison between the signals captured by the various sensors may allow for detecting the position of a different talker and if so a beam of sensitivity towards that talker may also be formed in order to capture and analyze their voice. Finally the signal captured by at least one of these sensors may be used to measure the type and loudness of the surrounding noise that the user is exposed to. These beams are created on the processor of the ear-piece, and sent as a stream of up to 3 audio channels from each ear to the detection device where the analysis of these audio channels will be analyzed to derive the emotional state of the user. In some cases, the user can use the app of the interface device to start the streaming/recording on the earpiece. Alternatively, some or all of these streams can be recorded automatically at intervals previously approved or set by the user.

FIG. 6 shows a flow diagram of an analysis process by comparing input data and calibration data by using a Siamese network 20. Calibration data 11, in this case a video and/or audio clip, is embedded in a reference branch 13 of a convolutional neural network 15. An input data 12, in this case a video or audio clip, is embedded in a difference branch 14 of the convolutional neural network 15. The Siamese network 20 ensures, that calibration data 11 and input data 12 share the same weights 16 respectively, this guarantees that each audio and video clip is embedded in their respective representation space. Of the embedded data, emotional features 17, 18 are extracted and a distance 19 between the emotional feature 17 and the emotional feature 18 is calculated.

FIG. 6 shows a diagram of the mode of operation of a self-supervised learning computer program structure 66 for learning audio-visual features.

The diagram shows a first video clip 60, a second video clip 61, and an nth video clip 62 on the left side. On the right side, the diagram shows a first audio clip 63, a second audio clip 64, and an nth audio clip 65. N represents a certain amount of, in this case 3-5 sets of audio and video clips.

The first video clip 60 and the second audio clip 64 are a set, the audio clip 64 matches the video clip 60. The second video clip 61 and the nth audio clip 65 are a set, as well as the nth video clip 62 and the first audio clip 63.

The self-supervised learning structure comprises a self-supervision signal that mixes sets of audio and video clips and learns to match the sets. The self-supervision signal used to learn useful features is the classification task of learning to predict a correspondence between audio and face video clips ie., learning to predict which video matches with which audio. So, the self-supervised learning structure is fed n, in this case 3-5, sets of matching audio and video clips, and learns to predict a correlation between the audio and video clips and thus, learns to match video clip 60 to audio clip 64, video clip 61 to the nth audio clip 65 and so on.

Learning to predict this correlation can directly lead to networks learning to focus on facial micro-expressions and audio tonality.

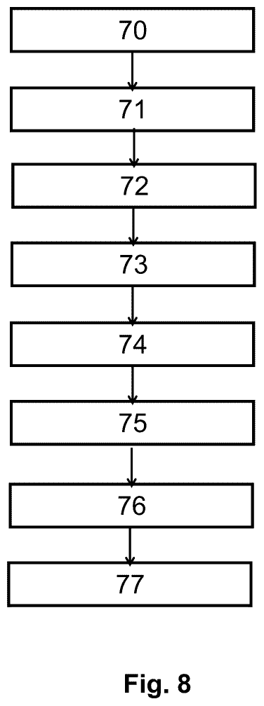

FIG. 8 shows the flow of a computer-implemented method for self-supervised audio-visual feature learning. The method uses a Siamese neural network for ensuring, that each video and audio clip is embedded in its own respective space in an emotional landscape.

The method uses two batches of sets of audio and video clips to train a self-supervised audio-visual feature learning computer program structure. The audio and video clips are mixed and a match between audio and video clips is predicted to train the computer program structure in recognizing audio-visual features.

In the first step 70, the first batch of sets of matching audio and video clips of multiple subjects is fed into the Siamese neural network. These audio-video clips are taken from the Google Audioset. In a batch for training the deep neural network are between 3-5 subjects. The total number may vary from a hundred to tens of thousands of subjects.

In a second step 71, the audio clips are separated from the video clips

The computer program structure comprises a proxy task for mixing and matching the audio and video clips. So, that in a third step the audio clips are mixed and the video clips are mixed, and in a fourth step 73 each audio clip is predicted to match a video clip.

The use of the first batch allows to train the self-supervised audio-visual feature learning program structure to be robust to changes in microphone, video camera, position of the camera, background noise etc. and to build a model that is robust in generalizing across devices and across cultures and generates robust features for both audio and video modes.

Then, in a fifth step 74, the second batch of sets of matching video and audio data of a singular subject is fed into the Siamese neural network. The self-supervised learning structure is fed videos from public emotion datasets like Ravdess, IEMOCAP to feed 5 videos of different emotions from the same subject as input samples to the Siamese neural network.

For the second batch, the proxy task is used again, so that in a sixth step 75 the audio clips of the second batch are separated from the video clips of the second batch and in a seventh step 76 these audio clips are mixed and the video clips are mixed. Then, in an eighth step 77, each audio clip of the second batch is predicted to match a video clip of the second batch.

The use of the second batch forces the network to hone in on what separates different audio and video from the same subject—their expressions and tone.

This method leads the self-supervised learning structure to build representations highly useful for embedding and recognizing emotions.

1-19. (canceled)

20. A detection device for detecting the emotional state of a user comprising
a processing unit for processing data,
a main data storage unit for storing data,
a connecting element for connecting the detection device to an interface device,
wherein the detection device is adapted to be calibrated to said user by use of the processing unit and calibration data, and wherein the processing unit is adapted to analyze input data based on said calibration.

21. The detection device according to claim 20, wherein the processing unit is adapted to process said input data and said calibration data by preparing the input data and the calibration data and extracting emotions features of said input data and said calibration data.

22. The detection device according to claim 21, wherein the processing unit comprises a deep neural network, wherein the processing unit is adapted to embed the extracted emotions features of the input data and the calibration data into the deep neural network and to create an emotional landscape of the user.

23. The detection device according to claim 20, wherein the processing unit is adapted to calculate an emotional state based on multiple input data and multiple comparisons of said input data to said calibration data.

24. The detection device according to claim 20, wherein the detection device comprises a self-supervised learning computer program structure for learning to extract meaningful features of data of the user for emotion detection, the self-supervised learning computer program structure is adapted to learn to predict matching audio and video data of the user.

25. The detection device according to claim 20, wherein at least one of the main data storage unit and an additional temporary storage unit is adapted for temporarily storing the input information.

26. The detection device according to claim 20, wherein the connection device comprises a wired or wireless connection element.

27. A system for detecting the emotional state of a user comprising a detection device according to claim 20 and at least one recording device for sensing the user.

28. The system according to claim 27, wherein the system is adapted to be calibrated to the user by use of the processing unit and calibration data recorded by the recording device.

29. The system according to claim 27, wherein the system comprises an ear-piece, wherein the recording device is part of this ear-piece.

30. The system according to claim 29, wherein the ear-piece comprises at least one of a speaker and one or more additional recording devices.

31. The system according to claim 27, wherein the system comprises an interface device for interaction between the system and the user, the interface device being connected to the detection device by the connecting element.

32. The system according to claim 27, wherein the system comprises multiple recording devices, wherein the recording devices comprise at least one of an acceleration sensor and a temperature sensor and a humidity sensor.

33. A computer-implemented method for detecting an emotional state of a user, comprising the steps of
providing at least one of a detection device and a system, a recording device and an interface device,
recording calibration data of the user by use of the recording device as input information in the main storage unit or the temporary storing unit,
calibrating the detection device to the user by processing the calibration data by a processing unit of the detection device,
recording input data by the recording device,
analyzing the input data by the processing unit by comparing input data to the reference data,
determining a commonality of the input data and the reference data.

34. The method according to claim 33, comprising the steps of
preparing calibration data
preparing input data,
wherein the calibration data and the input data is video data of the user and/or audio data of the user, wherein the preparing includes at least one of
extracting bounding boxes to detect frontal faces, and
cropping video data and
resizing video data and
splitting audio data into time windows of a certain length and
splitting audio data into frames.

35. The method according to claim 34, comprising the steps of
extracting emotional features of the calibration data,
extracting emotional features of the input data,
wherein an emotional feature extracted is at least one of
a fundamental frequency of the voice
a formant frequency of the voice
jitter of the voice
shimmer of the voice
intensity of the voice.

36. The method according to claim 33, comprising the steps of
recording one or multiple, input data of the user,
determining an emotional state of the user,
outputting the determined emotional state on the interface device.

37. The method according to claim 33, wherein the method comprises the steps of
providing a Siamese neural network,
feeding in a first batch of matching audio and video data of multiple subjects, the audio and video data are separated from each other, into the Siamese neural network,
predicting a correlation between said audio data and said video data of the first batch,
feeding in a second batch of matching video and audio data separately of a single subject concerning different emotional states of the subject into the Siamese neural network,
predicting a correlation between said audio data and said video data of the second batch.

38. A computer program product comprising instructions which when the program is executed by a computer processing unit of a detection device of claim 20 cause the computer to carry out method steps for detecting an emotional state of a user, comprising the steps of
providing at least one of a detection device and a system, a recording device and an interface device,
recording calibration data of the user by use of the recording device as input information in the main storage unit or the temporary storing unit,
calibrating the detection device to the user by processing the calibration data by a processing unit of the detection device,
recording input data by the recording device,
analyzing the input data by the processing unit by comparing input data to the reference data,
determining a commonality of the input data and the reference data.
